(19) United States
(12) Patent Application Publication
(10) Pub. No.: US 2004/0187877 A1
(43) Pub. Date: Sep. 30, 2004

(54) Method for repair of liver tissue

(76) Inventors: Stephen F. Badylak, West Lafayette, IN (US); Sangeeta N. Bhatia, LaJolla, CA (US)

Correspondence Address: BARNES & THORNBURG, 11 SOUTH MERIDIAN, INDIANAPOLIS, IN, 46204

(21) Appl. No.: 10/728,291

(22) Filed: Dec. 4, 2003

Related U.S. Application Data
(60) Provisional application No. 60/431,091, filed on Dec. 4, 2002.
(60) Provisional application No. 60/444,092, filed on Jan. 31, 2003.

(51) Int. Cl. A61F 2/02

(52) U.S. Cl. 128/898; 623/902

(57) ABSTRACT

A method for inducing the repair of damaged or diseased liver tissue in vivo is provided. The method comprises the step of administering to the patient a graft composition comprising basement membrane tissue of a warm-blooded vertebrate in an amount effective to induce the repair of the liver tissue at the site of administration of the graft composition.

### FIELD OF THE INVENTION

The present invention relates to a method for inducing the repair of damaged or diseased liver tissue. More particularly, the present invention is directed to the use of a non-immunogenic tissue graft composition comprising basement membrane to induce the repair of damaged or diseased liver tissue in vivo.

### BACKGROUND AND SUMMARY

There has been much research effort directed to finding natural and synthetic materials having the requisite properties for use as tissue grafts. Intestinal submucosa tissue can be used in a number of tissue graft applications including enhancing wound healing, promoting endogenous tissue growth, stimulating cell proliferation, and inducing cell differentiation. It has been found that basement membranes (stroma) prepared from liver tissue of warm-blooded vertebrates by removing cellular components of the liver tissue exhibit certain mechanical and biotropic properties similar to those which have been reported for intestinal submucosal tissue. See U.S. Pat. Nos. 4,902,508, 5,281,422, and 5,275,826. However, liver basement membrane is an extracellular matrix that is structurally distinct from submucosa extracellular matrices.

Although the liver plays a central role in numerous regulatory processes in the body, including glucose metabolism, insulin regulation, anabolic processes for the musculo-skeletal system and the central nervous system, and the maintenance of appropriate levels of circulating proteins essential for homeostasis, tissue grafts to promote the growth and repair of liver tissue have not previously been developed. Accordingly, in one embodiment, basement membranes are used as a non-immunogenic tissue graft composition for the repair of damaged or diseased liver tissue in vivo. The method comprises the step of administering to a patient a graft composition comprising basement membrane tissue of a warm-blooded vertebrate in an amount effective to induce the repair of the liver tissue at the site of administration of the graft composition.

In one embodiment, the basement membrane tissue graft composition comprises the basement membrane of liver tissue of a warm-blooded vertebrate, for example, liver basement membrane, substantially free of cells (e.g., hepatocytes and bile duct cells) of the warm-blooded vertebrate. The basement membrane graft composition can be implanted, or can be fluidized and injected, into a vertebrate host to contact damaged or diseased liver tissues and to induce the repair or replacement of the damaged or diseased liver tissues.

### BREIF DESCRIPTION OF THE DRAWINGS

FIG. 1 shows a comparison of albumin production for hepatocytes grown on a double gel substrate (gel 1-3), on liver basement membrane (ibm 1-3), and on adsorbed collagen (col 1-3).

FIG. 2 shows a comparison of urea production for hepatocytes grown on a double gel substrate (circles), on liver basement membrane (triangles), and on adsorbed collagen (squares).

FIG. 3 shows a comparison of DNA measured from hepatocytes grown on adsorbed collagen (bar 1), a double gel substrate (bar 2), and liver basement membrane (bar 3).

FIG. 4 shows a comparison of resorufin activity (reflects cytochrome P450 activity) for hepatocytes grown on a double gel substrate (bar labeled “gel” in FIG. 4), liver basement membrane (bar labeled “LBM” in FIG. 4), or on adsorbed collagen (bar labeled “col” in FIG. 4).

FIG. 5 shows the macroscopic and microscopic features of LBM. (A) Brightfield photograph of rehydrated LBM, 1×; (B) SEM image of LBM magnified 2,200×; and (C) Brightfield photograph of remodeled LBM after 35 days of culture. Viable hepatocytes are stained with MTT (precipitate).

FIG. 6 shows primary rat hepatocyte morphology on LBM after 7 days of culture. (A) SEM images of cells at 850×; (B) 3200 ×magnification; and (C) fluorescently-labeled hepatocytes and autofluorescent LBM at 20×.

### DETAILED DESCRIPTION

The tissue graft composition comprises basement membrane prepared by separating the basement membranes from the natively associated cellular components of tissue of a warm-blooded vertebrate. According to one embodiment, the preparative techniques described below provide an extracellular matrix composition comprising liver basement membrane substantially free of cellular components (e.g., hepatocytes and bile duct cells). These compositions are referred to herein generically as liver basement membrane (LBM). Other organ tissue sources of basement membrane for use in accordance with this invention include spleen, lymph nodes, salivary glands, prostate, pancreas and other secreting glands.

Basement membranes can be prepared from tissue harvested from animals raised for meat production, including, for example, pigs, cattle and sheep or other warm-blooded vertebrates. Thus, there is an inexpensive commercial source of tissue for preparation of the tissue graft compositions for use in accordance with the present invention.

In accordance with one embodiment, a graft composition comprising an extracellular matrix comprising liver basement membranes is prepared from liver tissue of a warm-blooded vertebrate. In one embodiment, the graft composition comprises liver basement membrane separated from endogenous cells associated with the source vertebrate liver tissue used to prepared the composition.

This graft composition is useful as a non-immunogenic tissue graft capable of inducing the repair of liver tissue when implanted in the liver of a warm-blooded vertebrate. In one embodiment, repair of liver tissue includes, for example, the repair, replacement, regeneration, growth, and differentiation of liver tissue. In another embodiment repair of liver tissue can also include an increase in the rate of healing of liver tissue or maintenance of the phenotypic stability of liver tissue. In another embodiment, repair can be induced in a damaged or diseased liver wherein damaged liver tissue includes, for example, liver tissue in a patient with compromised liver function and liver tissue in a healthy donor, for example, who has donated healthy liver tissue to a patient with compromised liver function in need thereof.

The preparation of liver basement membrane from the liver tissue of a warm-blooded vertebrate can be carried out by removing the cellular components from liver tissue. The process is carried out to separate the cells from the basement membranes without damaging, or at least with minimal disruption or damage to, the basement membrane tissue. Removal of the cellular components from the liver extracellular matrix allows the preparation of a graft composition that is non-immunogenic, when the graft composition is implanted in the patient. Liver basement membranes can be prepared from warm-blooded vertebrate liver tissue by treating the liver tissue with a cell dissociation solution for a period of time sufficient to release the cellular components of the liver tissue from the extracellular components without substantial disruption of the extracellular components, and by separating the cellular components from the extracellular components. The cell dissociation solution can be, for example, a chaotropic agent, an enzyme, or combinations of these agents.

The first step in preparing LBM in accordance with one method of preparing LBM comprises slicing a segment of liver tissue into pieces (e.g., into strips or sheets) to increase the surface area-to-volume ratio of the liver tissue. In one embodiment the liver tissue is sliced into a series of sheets each having a thickness of about 50 to about 500 microns, or about 250 to about 300 microns. Freshly harvested liver tissue can be sliced using a standard meat slicer, or the tissue can be frozen and sliced with a cryomicrotone. The thin pieces of liver tissue can then be treated with a solution that releases component liver cells from the extracellular basement membrane matrix.

In accordance with one embodiment, the liver tissue is treated with a solution comprising an enzyme, for example, a protease, such as trypsin or pepsin. Because of the collagenous nature of the basement membranes and the desire to minimize degradation of the basement membrane structure during cell dissociation, collagen specific enzyme activity should be minimized in the enzyme solutions used in the cell-dissociation step. In addition, the liver tissue can also be treated with a calcium chelating agent or chaotropic agent such as a mild detergent (e.g., Triton X-100). Thus, in one embodiment liver tissue can be treated by suspending slices or strips of the liver tissue in a cell-dissociation solution containing enzyme(s) and chaotropic agent(s). However, the cell dissociation step can also be conducted using a calcium chelating agent or a chaotropic agent in the absence of enzymatic treatment of the tissue.

In one embodiment, the cell-dissociation step is carried out by suspending liver tissue slices in a solution containing about 0.05 to about 2%, more typically about 0.1 to about 1% by weight of a protease, optionally containing a chaotropic agent or a calcium chelating agent in an amount effective to optimize release and separation of cells from the liver basement membrane without substantial degradation of the liver basement membrane matrix.

After contacting the liver tissue with the cell-dissociation solution for a time sufficient to release the cells from the liver basement membrane matrix, the resulting liver basement membrane can be rinsed one or more times with saline and optionally stored in a frozen hydrated state or a partially dehydrated state until used as described below. The cell-dissociation step may require several treatments with the cell-dissociation solution to release the cells from the liver basement membrane. In one embodiment, liver tissue is treated with a protease solution to remove the associated cells, and the resulting liver basement membrane can be further treated to remove or inhibit any residual enzyme activity. For example, the resulting liver basement membrane can be heated or treated with one or more protease inhibitors.

In another embodiment, the preparation of liver basement membrane from liver tissue of a warm-blooded vertebrate can be carried out by removing cells, cellular components, and other components, such as endotoxin and DNA, from liver tissue. In general, according to this embodiment, liver basement membrane is prepared by a method comprising the steps of protease digestion and treating the liver tissue with a non-denaturing detergent followed by treatment with a denaturing detergent for a period of time sufficient to release cells, cellular components, and other components, such as endotoxin and DNA, from the extracellular matrix without substantial disruption of the extracellular matrix, and separating the dissociated components from the extracellular matrix. Typically the liver tissue is sliced into sheets or strips having a thickness of up to about 2000 μm before subjecting the liver tissue to protease digestion.

The first step in preparing LBM can comprise slicing a segment of fresh or frozen liver tissue into pieces (e.g., strips or sheets) to increase the surface area-to-volume ratio of the liver tissue. In one embodiment, the liver tissue is sliced into a series of sheets each having a thickness of about 50 to about 2000 microns, or about 100 to about 1000 microns, or about 200 to about 600 microns. Freshly harvested liver tissue can be sliced using a standard meat slicer, or the tissue can be frozen and sliced with a meat slicer or cryomicrotone. In one embodiment, prior to slicing, the liver can be separated into lobes, trimmed, cut into uniform rectangular pieces, and can be frozen.

Before contacting the liver tissue with the protease-containing solution for a time sufficient to release cells, cellular components such as DNA, and endotoxin from the matrix, the liver sheets or strips can be rinsed one or more times, such as with deionized water, saline, or a buffered solution and optionally stored in a frozen hydrated state or a partially dehydrated state until used as described below. For example, the liver sheets or strips could be rinsed three times for 30 minutes each with deionized water, saline, or a buffer. Alternatively, the liver slices can be treated with the protease-containing solution without prior rinsing.

The deionized water, saline, or buffer can then be strained from the liver slices, for example, using a sieve, and hepatocytes and hepatocyte cell fragments can be mechanically dissociated from the liver basement membrane. For example, the liver slices can be massaged on a screen or ultrasound can be used to dissociate cells and cell components from the liver basement membrane. This step also hastens lysis of hepatocytes, and if this step is performed, it is done carefully so that the liver slices are not torn.

The thin slices of liver tissue can then be contacted with an aqueous composition containing a protease to partially hydrolyze the liver tissue and release liver cells and other components from the extracellular basement membrane matrix. In accordance with one embodiment, the liver tissue is contacted with an aqueous composition comprising an enzyme, for example, a protease, such as trypsin. Other proteases suitable for use in accordance with the invention include pepsin, bromelain, papain, chymotrypsin, lysosomal proteases, cathepsin, alcalase, savinase, chymopapain, clostripain, endoproteinase Asp N, protease V8, proteinase K, subtilisin proteases, thermolysin, plasmin, and pronase. Combinations of proteases can also be used. Because of the collagenous structure of the liver basement membrane and the desire to minimize degradation of the membrane structure during cell dissociation, collagen specific enzyme activity should be minimized in the enzyme compositions used in the protease digestion step.

The liver tissue is typically also contacted with a calcium chelating agent, such as EDTA, concurrently with the protease treatment. Thus, in one embodiment liver tissue is treated by suspending slices or strips of the tissue in a solution containing a protease and EDTA. As an alternative to a protease, the liver tissue can be contacted with any other enzyme that promotes cell dissociation without degrading the basement membrane structure, such as a GAGase, or the liver tissue can be treated with a combination of enzymes. In another embodiment, the liver tissue can be perfused with a protease solution with or without a Ca⁺⁺ chelating agent prior to slicing and after slicing.

In one embodiment the protease digestion step is carried out by contacting liver tissue slices with a solution, optionally with agitation, containing about 0.005 % of the protease (e.g., trypsin) by weight to about 2% of the protease by weight, more typically about 0.01 % of the protease by weight to about 1% of the protease by weight and containing a calcium chelating agent, such as EDTA, in an amount effective to optimize release and separation of cells and other components from the liver basement membrane without substantial degradation of the membrane matrix. The concentration of the calcium-chelating agent (e.g., EDTA) is typically about 0.01% of the calcium chelating agent by weight to about 2% of the calcium chelating agent by weight, preferably about 0.02% of the calcium chelating agent by weight to about 1% of the calcium chelating agent by weight. The protease digestion step is preferably carried out with heating, typically at about 37° C. The rinsing and mechanical dissociation steps described above can be repeated after the protease digestion step. Alternatively, mechanical dissociation, for example with ultrasound, can be performed during and/or after the protease digestion step.

The liver slices can then be contacted with a solution containing a non-denaturing detergent. This step is preferably carried out at room temperature, and optionally with agitation. The non-denaturing detergent is preferably Triton X-100, typically a Triton X-100 solution of about 0.5% to about 5%, more typically about 2% to about 4%. However, any non-denaturing detergent known in the art which is effective to release cells and other components from the liver basement membrane without substantial disruption of the basement membrane matrix can be used.

Exemplary of non-denaturing detergents that can be used are polyoxyethylene ethers, 3-[(3-cholamidopropyl dimethylammonio]-1-propane-sulfonate (CHAPS), nonylphenoxy polyethoxy ethanol, polyoxyethylenesorbitans, sodium lauryl sarcosinate, and alkyl glucosides including C₈-C₉ alkyl glucoside. Various types of nonylphenoxy polyethoxy ethanol detergents are available including NP-4, NP-7, NP-9, NP-10, NP-35, and NP-40, sold under the trademark Niaproof® (Niacet Corp.), and any of these types, or any other suitable types of this surfactant, can be used. Polyoxyethylene ethers include Triton X-100, Triton X-114, Triton X-405, Triton N-101, Triton N-42, Triton N-57, Triton N-60, Triton X-15, Triton X-35, Triton X-45, Triton X-102, Triton X-155, Triton X-165, Triton X-207, Triton X-305, Triton X-705-70, and Triton B-1956, Triton CG-110, Triton XL-80N, and Triton WR-1339. Any of these polyoxyethylene ethers or other suitable forms can be used. Polyoxyethylenesorbitans that can be used include Tween 20, Tween 21, Tween 40, Tween 60, Tween 61, Tween 65, Tween 80, Tween 81, Tween 85, and Span 20.

The rinsing steps described above can be repeated after contacting the liver slices with the non-denaturing detergent to remove most, if not all, of the non-denaturing detergent. This step prevents the non-denaturing detergent from interfering with the activity of the denaturing detergent in the subsequent detergent extraction step. The mechanical dissociation steps can be repeated as needed.

After treatment with the non-denaturing detergent, the liver slices can be contacted with a solution containing a denaturing detergent. This step is preferably carried out at room temperature and optionally with agitation. The denaturing detergent is preferably deoxycholate, typically a deoxycholate solution of about 0.5% to about 8%, more typically about 2% to about 5%. However, any denaturing detergent known in the art which is effective to release cells and other components from the liver basement membrane without substantial disruption of the basement membrane matrix can be used including such denaturing detergents as sodium dodecylsulfate. The purified LBM can then be thoroughly rinsed as described above to remove as much residual detergent as possible and the LBM can be stored (e.g., in deionized water at 4° C.) until further use or can be used immediately following the purification procedure.

The protease digestion step and the treatments with the non-denaturing and denaturing detergents can be performed one or more times to release the cells and other components described above from the basement membrane. Additionally, the rinsing steps can be performed one time or multiple times and the mechanical dissociation steps can be repeated as needed or may not be performed if visual inspection indicates that a step to promote mechanical dissociation of cells or other cell components is not required. Moreover, the concentration of the protease and the concentrations of the non-denaturing and denaturing detergents can be varied depending on the thickness of the liver slices used and the specific protease and detergents used in the purification protocol.

Basement membranes can be fluidized (converted to an injectable form) in a manner similar to the preparation of fluidized intestinal submucosa, described in U.S. Pat. No. 5,275,826, the disclosure of which is incorporated herein by reference. Basement membranes (separated from cells from the source tissue) can be comminuted by tearing, cutting, grinding, shearing and the like. The basement membranes can be ground in a frozen or freeze-dried state is preferred although good results can also be obtained by subjecting a suspension of basement membrane to treatment in a high speed, high shear blender and dewatering, if necessary, by centrifuging and decanting the excess water. Additionally, the comminuted fluidized tissue can be solubilized by enzymatic digestion with a protease, for example, with a collagenase or another appropriate enzyme, such as a glycanase, or another enzyme that disrupts the matrix structural components, for a period of time sufficient to solubilize the tissue and to form a substantially homogeneous solution. The viscosity of fluidized tissue can be manipulated by controlling the concentration of the basement membrane component and the degree of hydration. The viscosity can be adjusted, for example, to a range of about 2 to about 300,000 cps at 25° C.

The use of powder forms of basement membrane is also contemplated. In one embodiment, a powder form of basement membrane is prepared by pulverizing basement membrane and freezing the tissue under liquid nitrogen to produce particles ranging in size from 0.1 to 1 mm². The particulate composition is then lyophilized overnight and sterilized to form a solid substantially anhydrous particulate composite. Alternatively, a powder form of basement membrane can be formed from fluidized basement membranes by drying the suspensions or solutions of comminuted basement membrane. The dehydrated forms can be rehydrated and used as tissue graft compositions without any apparent loss of their ability to promote growth and repair of liver tissue.

Basement membranes can also be extracted with guanidine hydrochloride and/or urea, as described in Example 5. Briefly, the powder form of basement membranes can be suspended in an extraction mixture containing 4M guanidine hydrochloride, 2M urea, and protease inhibitors and stirred vigorously. The extraction mixture can then be centrifuged and the supernatant removed and dialyzed extensively to further remove insoluble material. The supernatant can be used immediately or lyophilized and stored for later use.

The basement membrane graft compositions can be sterilized using conventional sterilization techniques including tanning with glutaraldehyde, formaldehyde tanning at acidic pH, ethylene oxide treatment, propylene oxide treatment, gas plasma sterilization, gamma radiation, and peracetic acid sterilization. A sterilization technique which does not significantly weaken the mechanical strength and biotropic properties of the basement membrane is preferably used. In one embodiment, basement membranes can be sterilized by exposing the graft composition to peracetic acid and/or low dose gamma irradiation and/or gas plasma sterilization. Basement membranes can be disinfected and sterilized through the use of peracetic acid and/or one megarad of gamma irradiation without adversely effecting the mechanical properties or biological properties of the tissue. Treatment with peracetic acid can be conducted at a pH of about 2 to about 5 in an aqueous ethanolic solution (2-10% ethanol by volume) at a peracid concentration of about 0.03 to about 0.5% by volume. After the graft composition has been sterilized, the graft composition can be wrapped in a porous plastic wrap and sterilized again using electron beam or gamma irradiation sterilization techniques.

In accordance with one embodiment, liver basement membrane is used as a tissue graft composition for inducing the repair of damaged or diseased liver tissue in a patient in need thereof. Such tissue graft compositions lend themselves to a wide variety of surgical applications relating to the repair or replacement of damaged liver tissues. Such tissue graft compositions are administered by surgical techniques known to those skilled in the art. Such surgical applications include repair of liver tissue (e.g., repair, replacement, regeneration, growth, or differentiation of liver tissue or an increase in the rate of healing of liver tissue or maintenance of the phenotypic stability of liver tissue) from both hepatic and non-hepatic sites (e.g., hematopoietic stem cells, pancreas, etc.).

In one embodiment, liver tissue can be tissue recognized in the art as having the architecture of art-recognized liver tissue, or liver tissue can be tissue that does not have the architecture of art-recognized liver tissue, but provides normal liver functions.

In one embodiment, the basement membrane tissue graft compositions are used advantageously to induce the formation of liver tissue at a desired site in a warm-blooded vertebrate. Compositions comprising a basement membrane extracellular matrix can be administered to a patient in an amount effective to induce liver tissue growth at a site in the patient in need of repair or regrowth due to the presence of damaged or diseased liver tissue. The present basement membrane-derived tissue graft compositions can be administered to the patient in either solid form, by surgical administration, or in powder or gel form, or in fluidized form or in the form of an extract, by, for example, injection in accordance with the procedures described for use of intestinal submucosa in U.S. Pat. Nos. 5,281,422 and 5,352,463, each expressly incorporated herein by reference.

In accordance with another embodiment of the invention, hepatocytes can also be grown in vitro on liver basement membrane to form liver tissue for use in drug discovery or drug development assays. In this regard, liver tissue grown in vitro on liver basement membrane can be used to test ADMET properties (i.e., adsorption, distribution, metabolism, excretion, and toxicity) of drugs.

The graft compositions used in accordance with this invention, undergo biological remodeling upon implantation. They serve as a rapidly vascularized matrix for supporting the growth of new liver tissue to promote the repair or replacement of damaged or diseased tissue. The basement membrane graft composition can be formed in a variety of shapes and configurations, for example, to serve as a graft for replacement of a portion of liver tissue or a patch for a tear in a patient's liver. The basement membranes can be layered or even multilayered. For example, the opposite end portions and/or the opposite lateral portions can be formed to have multiple layers of the graft material to provide reinforcement for attachment to physiological structures, such as liver tissue. The end portions or lateral portions of the basement membrane graft composition can be formed, manipulated, or shaped to be attached, for example, to liver tissue in a manner that will reduce the possibility of the graft tearing at the point of attachment. For example, the material can be folded to provide multiple layers for gripping, for example, with sutures, spiked washers, or staples. Alternatively, the basement membrane graft material can be folded to join the end portions or lateral portions to provide a reinforced graft material.

During preparation of the basement membranes, the tissue can be cut or sliced into pieces/slices. After the cell-dissociation processing step(s) the individual segments of basement membrane can be overlapped (e.g., laid over each other or having a portion overlapped) with one another and bonded together using standard techniques known to those skilled in the art, including the use of sutures, crosslinking agents, and adhesives or pastes. Alternatively, in one embodiment, the overlapped layers of basement membrane are fused to one another by applying pressure to the overlapped regions under dehydrating conditions, including any mechanical or environmental condition which promotes or induces the removal of water from the basement membrane tissue. To promote dehydration of the compressed basement membrane tissue, at least one of the two surfaces used to compress the tissue can be water permeable. Dehydration of the tissue can optionally be further enhanced by applying blotting material, heating the tissue or blowing air across the exterior of the compressing surfaces. Accordingly, multilayer basement membrane graft constructs can be prepared to provide basement membrane graft compositions of enhanced strength.

In addition, by overlapping a portion of one piece of basement membrane with a portion of at least one additional piece of basement membrane and bonding the overlapped layers to one another, large area sheets of basement membrane can be formed. In one embodiment, during formation of the large area sheets of tissue, pressure is applied to the overlapped portions under dehydrating conditions by compressing the overlapped tissue segments between two surfaces. The two surfaces can be formed from a variety of materials and in any shape depending on the desired form and specification of the basement membrane graft construct. The two surfaces used for compression can be formed as flat plates but they can also include other shapes such as screens, opposed cylinders or rollers, and complementary nonplanar surfaces. Each of these surfaces can optionally be heated or perforated (e.g., at least one of the two surfaces can be water permeable including surfaces that are water absorbent, microporous or macroporous (e.g., including perforated plates or meshes made of plastic, metal, ceramics or wood)).

The basement membrane can be compressed in accordance with one embodiment by placing the overlapped portions of the strips of cell-dissociated basement membrane on a first surface and placing a second surface on top of the exposed basement membrane surface. A force can then be applied to bias the two surfaces towards one another, compressing the basement membranes between the two surfaces. The biasing force can be generated by any number of methods known to those skilled in the art including the passage of the apparatus through a pair of pinch rollers (the distance between the surface of the two rollers can be less than the original distance between the two plates), the application of a weight on the top plate, the use of a hydraulic press or the application of atmospheric pressure on the two surfaces, and the like.

In one embodiment, a multi-layered basement membrane graft composition is prepared without the use of adhesives or chemical pretreatments by compressing at least the overlapped portions of basement membrane tissue under conditions that allow dehydration of the material concurrent with the compression of the tissue. To promote dehydration of the compressed material, at least one of the two surfaces (e.g., a plate) used to compress the tissue is water permeable. Dehydration can optionally be further enhanced by applying blotting material, heating the graft material or blowing air across the exterior of the two surfaces used for compression. The compressed multi-layered basement membrane material can be removed from the two surfaces as a unitary compliant large area graft construct. The construct can be further manipulated (e.g., cut, folded, sutured, and the like) to suit various surgical applications where the basement membrane material is required.

In accordance with one embodiment, the basement membrane graft composition comprises multiple layers of basement membrane comprising 2-12 layers of basement membrane, more preferably 4-6 layers. The multi-layered composition in one embodiment comprises partially overlapped strips of basement membrane and more preferrably the tissue graft composition is formed as a multilayered homolaminate (i.e., having the same number of layers throughout the graft) construct.

A vacuum can optionally be applied to the basement membranes during the compression procedure. The applied vacuum enhances the dehydration of the tissue and may assist the compression of the tissue. Alternatively, the application of a vacuum can provide the sole compressing force for compressing the overlapped portions of the multiple layers of basement membranes. For example, in one embodiment the overlapped basement membrane is laid out between two surfaces, preferably one of which is water permeable. The apparatus is covered with blotting material, to soak up water, and a breather blanket to allow air flow. The apparatus is then placed in a vacuum chamber and a vacuum is applied, for example, ranging from 35.6-177.8 cm of Hg (0.49-2.46 Kg/cm²). In one embodiment, approximately 129.5 cm of Hg (1.76 Kg/cm²) is applied. Optionally a heating blanket can be placed on top of the chamber to heat the basement membrane composition during compression. Chambers suitable for use in this embodiment are known to those skilled in the art and include any device that is equipped with a vacuum port. The resulting drop in atmospheric pressure coacts with the two surfaces to compress the basement membrane tissue and simultaneously dehydrate the compressed tissue.

In another embodiment, the basement membrane graft compositions can be formed from fluidized forms of basement membrane that is gelled to form a solid or semi-solid matrix. Gels can be prepared from digest solutions by adjusting the pH of such solutions to about 6.0 to about 7.4.

In one embodiment, basement membrane is capable of inducing liver tissue remodeling and regeneration upon implantation in vivo. In one embodiment, the liver tissue replacement capabilities of graft compositions comprising basement membrane of warm-blooded vertebrates are further enhanced or expanded by seeding the basement membranes with cells prior to implantation. For example, a basement membrane-derived graft composition can be seeded with cells such as hepatocytes, endothelial cells, smooth muscle cells, and the like. The cells can be expanded, using cell culture conditions known in the art, prior to implantation of the graft composition into the patient or the graft composition with the added cells can be implanted without expansion of the cells. The basement membrane graft compositions of the present invention can also be combined with, for example, peptides, proteins, or glycoproteins that facilitate cellular proliferation, such as laminin and fibronectin and growth factors such as epidermal growth factor, platelet-derived growth factor, transforming growth factor beta, or fibroblast growth factor. Basement membranes can also serve as a delivery vehicle, in fluidized form, gel form, powder form, extract form, or in its native solid form, for introducing various cell populations, including genetically modified cells, into liver tissue in a patient.

In another embodiment, compositions comprising basement membranes and, optionally, added cells and/or other factors can be encapsulated in a biocompatible matrix for implantation into a patient. The encapsulating matrix can be configured to allow the diffusion of nutrients to the encapsulated cells while allowing the products of the encapsulated cells to diffuse from the encapsulated cells to the patient's cells. Suitable biocompatible polymers for encapsulating living cells are known to those skilled in the art. For example a polylysine/alginate encapsulation process has been previously described by F. Lim and A. Sun (Science, vol. 210, pp. 908-910). Indeed, the present basement membrane composition itself could be used advantageously to encapsulate cells in accordance with this invention for implantation into a patient.

### EXAMPLE 1

Liver Basement Membrane Preparation

Porcine livers were collected and were transported on ice. For each liver, the four lobes were separated using a scalpel/scissors/razor blade and each lobe was trimmed to a fairly uniform rectangular shape. If the liver was to be frozen prior to further processing, each lobe was trimmed and wrapped in a plastic bag and stored in the freezer.

Previously prepared (fresh or frozen) liver lobes were cut using a meat slicer. For cutting, the meat slicer was set to a setting of 1.0 (results in a slice thickness of about 50 microns) and the initial outer layers of the liver membrane were removed by cutting and discarded. Once the outer layers were removed, the meat slicer was set to a setting of 3.0 (results in a slice thickness of about 2000 microns) and the liver slices were cut into slices of uniform thickness. The liver slices were maintained at 4° C. during the cutting process and were stored in the freezer until needed or were used immediately.

Prior to purification (i.e., decellularization), the slices of liver were trimmed with a scalpel/razor blade to remove any remnants on the outer edge of the liver slices from the slicing process. If thickness readings were taken, digital calipers were used and the slices were measured while still frozen. To measure the thickness of the liver slices, the thickness of two small pieces of acrylic was measured using the calipers and the thickness was recorded. A frozen slice of liver was then placed between the acrylic pieces and the combined thickness was measured. The measurements were taken in several areas to get an average liver-acrylic combined thickness. The original thickness of the acrylic pieces was subtracted from the average combined liver-acrylic thickness to obtain the thickness of the liver slices. Generally, the liver slices ranged from about 50 μ to about 2000 μ in thickness.

Solutions for liver basement membrane purification were prepared as follows:

1. 3% (v/v) Triton X-100—For a 500 ml rinse, 15 ml of the concentrated Triton X-100 was added to 485 ml of deionized water. The Triton X-100 is viscous, so it was necessary to do a repeated backwashing of the graduated cylinder to remove residual Triton X-100. The Triton X-100 solution was mixed on a shaker to thoroughly dissolve the detergent in water.

2. 4% (w/v) Deoxycholic Acid—For a 500 ml rinse, 20 g of deoxycholic acid was added to 480 ml of deionized water and the solution was mixed until thoroughly dissolved.

3. 0.02% Tryrpsin/0.05% EDTA—Trypsin is commonly packaged at a concentration of 25 g/L. Therefore, for a 0.02% solution of trypsin in 500 ml, 0.1 grams of trypsin is required (equivalent to 4 ml of the concentrated trypsin/EDTA solution per 500 ml of deionized water). EDTA (.05%) is obtained by adding 0.25 g of EDTA (4 ml of trypsin/EDTA solution) to 495.75 ml of deionized water. The solution was agitated on a shaker to ensure adequate mixing.

In general, four liver slices were added per 1500 ml water bottle for each rinsing step, and 500 ml of rinse per 1500 ml water bottle was used. For the first wash, four trimmed liver slices were placed into a 1500 ml water bottle, and 1000 ml of deionized water was added to the water bottle(s). The bottle(s) were placed on a shaker for 30 minutes. After 30 minutes, the water was replaced with fresh deionized water and this process was repeated 2 times, for a total of three 30-minute rinses.

The deionized water was then strained from the liver slices using a sieve, and each liver slice was placed on a standard 12 inch by 12 inch aluminum window screen. Each liver slice was gently massaged by hand or using a rubber rolling pin to hasten the lysis of hepatocytes and to mechanically dissociate hepatocytes from the liver basement membrane. Care was taken to ensure that tears were not created in the slices. At this stage, all of the hepatocytes were not removed from the underlying liver basement membrane. The massaging step was repeated for each liver slice.

The liver slices were then returned in groups of four to the water bottles, and 500 ml of the 0.02% trypsin/0.05% EDTA solution was added to the water bottles. The liver slices were incubated in a 37° C. water bath for 1 hour. After one hour, the trypsin/EDTA solution was strained off using a sieve. Each slice was then momentarily rinsed under a stream of deionized water, and then the massaging step was repeated for each liver slice.

The liver slices were placed back into the bottles and 500 ml of the 3% Triton X-100 solution was added to the bottles. The bottles were placed on a shaker for 1 hour and were then briefly rinsed to remove the detergent solution. If necessary (as determined by visual inspection), the slices were massaged again.

The liver slices were then placed back into the bottles with 500 ml of 4% deoxycholic acid solution. The bottles were placed on the shaker for 1 hour. The purified liver basement membrane was thoroughly rinsed under deionized water for 3 to 5 minutes to remove as much residual detergent as possible. The purified liver basement membrane was stored in sterile deionized water at 4° C. until further use.

### EXAMPLE 2

Mechanical Properties of Purified Liver Basement Membrane

Porosity Index. Porosity of a graft material is typically measured in terms of ml of water passed per cm²min⁻¹ at a pressure of 120 mm Hg. The average porosity index of native LBM, purified as described above, was 1.7±1.2 (N=15). The average porosity indices for peracetic acid-treated LBM and peracetic acid and gamma-irradiated LBM, both purified as described above, were 4.3±2.1 (N=7) and 2.6±1.4 (N=7), respectively.

Suture Retention Strength. The suture retention strength test measures the force required to pull a suture through the material tested. The suture retention strength of native LBM (N=24), purified as described above, was approximately 0.45±0.14 Newtons (0.10±0.03 lbs.).

Ball Burst Testing. The ball burst test measures the force that a material can withstand. The ball burst strength of native LBM (N=3), purified as described above, was 19.66±4.27 Newtons (4.42±0.96 lbs.).

Thickness. The thickness of LBM (N=3), purified as described above, was 0.18±0.02 mm (0.0071±0.0008 inches).

### EXAMPLE 3

Preparation of Liver Basement Membrane

2 mM EDTA Chaotropic Solution Used In The Experiment

| | |
|---|---|
| 140 mM | NaCl |
| 5 mM | KCl |
| 0.8 mM | MgSO₄ |
| 0.4 mM | KH₂HPO₄ |
| 2 mM | EDTA |
| 25 mM | NaHCO₃ |

Procedure

Preparation of liver slices:

Liver frozen in −70° C. was sliced with a cryomicrotone to a thickness of about 50 μM. The slices of liver tissue were then subjected to enzymatic treatment (trypsin) with a chaotropic solution (samples 1 and 2), with enzyme alone (samples 3 and 4), or with a chaotropic solution alone (sample 5), as indicated below.

| Sample # | Treatment |
|---|---|
| 1) | 0.05% Trypsin in 2 mM EDTA solution |
| 2) | 0.1% Trypsin in 2 mM EDTA solution |
| 3) | 0.05% Trypsin in 2 mM PBS |
| 4) | 0.1% Trypsin in 2 mM PBS |
| 5) | 2 mM EDTA solution |

Liver slices were placed in five 50 ml tubes, each of which contained 25 ml of a different buffered enzyme treatment solution. The liver tissue was incubated at 37° C. in water bath with gentle shaking for 1 hour. The liver slices were washed twice with PBS with agitation/shaking for 1 hour at room temperature. The above enzymatic treatment steps were repeated three times.

The wash buffers were collected and spin them down in 2000 rpm for 10 min. The pellet was suspended and an equal amount of trypan blue was added to identify any remaining cells. The material was checked for presence of cells under microscope.

### EXAMPLE 4

Mechanical Properties of Isolated Liver Basement Membrane

Porosity of a graft material is typically measured in terms of ml of water passed per cm²min⁻¹ at a pressure of 120 mm Hg. The average “porosity index” established for two separate specimens of LBM prepared according to the procedure described in Example 3 was 1162. The suture retention strength of LBM is approximately 68 grams. The material appears to be anisotropic, with the suture strength being approximately the same in all directions.

### EXAMPLE 5

Preparation of Extracts of LBM

For fluidized or gel forms or for extracts of LBM, the tissue is stored in liquid nitrogen at −80° C. Frozen tissue is then sliced into 1 cm cubes, pulverized under liquid nitrogen with an industrial blender to particles less than 2 mm²and stored at −80° C. prior to use. Extraction buffers used for these studies included 4 M guanidine and 2 M urea each prepared in 50 mM Tris-HC1, pH 7.4. The powder form of LBM prepared by the method of Example 3 was suspended in the relevant extraction buffer (25% w/v) containing phenylmethyl sulphonyl fluoride, N-ethylmaleimide, and benzamidine (protease inhibitors) each at 1 mM and vigorously stirred for 24 hours at 4° C. The extraction mixture was then centrifuged at 12,000 ×g for 30 minutes at 4° C. and the supernatant collected. The insoluble material was washed briefly in the extraction buffer, centrifuged, and the wash combined with the original supernatant. The supernatant was dialyzed extensively in Spectrapor tubing (MWCO 3500, Spectrum Medical Industries, Los Angeles, Calif.) against 30 volumes of deionized water (9 changes over 72 hours). The dialysate was centrifuged at 12,000 ×g to remove any insoluble material and the supernatant was used immediately or lyophilized for long term storage.

Preparation of Fluidized Liver Basement Membrane

Partial digestion of the pulverized material (LBM was prepared by the method of Example 3) was performed by adding 5 g of powdered tissue to each 100 ml solution containing 0.1% pepsin in 0.5 M acetic acid and digesting for 72 hours at 4° C. Following partial digestion, the suspension was centrifuged at 12,000 rpm for 20 minutes at 4° C. and the insoluble pellet discarded. The supernatant was dialyzed against several changes of 0.01 M acetic acid at 4° C. (MWCO 3500). The solution was sterilized by adding chloroform (5 ml chloroform to each 900 ml 0.01 M acetic acid) to the dialysis LBM tissue reservoir. Dialysis of the LBM tissue was continued with two additional changes of sterile 0.01 M acetic acid to eliminate the chloroform. The contents of the dialysis bag were then transferred aseptically to a sterile container. The resultant fluidized composition was stored at 4° C.

Preparation of Liver Basement Membrane Gel Compositions

To prepare the gel form of LBM, 8 mls of fluidized LBM (prepared by the method of Example 5) was mixed with 1.2 ml 10×PBS Buffer (10×phosphate buffered saline containing 5 mg/L phenol red); 0.05 N NaOH (approx. 1.2 ml) was added to shift the pH to >8 and then 0.04 N HCI (approx 1.6 ml) was added to adjust the pH to between 6.6 and 7.4. The final volume was adjusted to 12 ml with water.

### EXAMPLE 6

Preparation of Liver Basement Membrane Powder

The use of powder forms of liver basement membrane is also contemplated. A powder form of liver basement membrane was prepared by pulverizing liver basement membrane under liquid nitrogen to produce particles ranging in size from 0.1 to 1 mm². The particulate composition was then lyophilized overnight and sterilized to form a solid substantially anhydrous particulate composite. Alternatively, a powder form of liver basement membrane can be formed from fluidized liver basement membranes by drying the suspensions or solutions of comminuted liver basement membrane.

### EXAMPLE 7

Albumin Assay

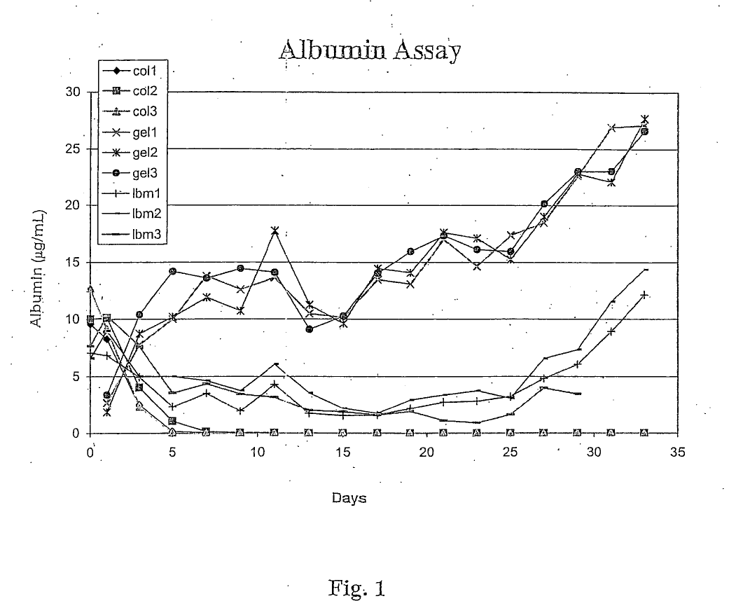

Hepatocytes were isolated and cultured as described in Biotechnol Prog., vol. 14, pp. 378-387 (1998). Hepatocyte culture medium was Dulbecco's Modified Eagle medium (DMEM, Gibco) supplemented with 10% fetal bovine serum (FBS, Sigma, St. Louis, Mo.), 0.5 U/ml of insulin, 7 ng/ml of glucagon, 20 ng/ml of epidermal growth factor, 7.5 mg/ml of hydrocortisone, 200 U/ml of penicillin, and 200 mg/ml of streptomycin. Hepatocytes were cultured in P-60 polystyrene tissue culture dishes between two collagen gel layers (double gel; gel 1-3 in FIG. 1), on liver basement membrane (Ibm 1-3 in FIG. 1), or on adsorbed collagen (col 1-3 in FIG. 1). Art-recognized procedures were used to culture hepatocytes on double gel substrates and on adsorbed collagen.

An albumin assay was performed on the hepatocytes grown on the various substrates as a marker of liver synthetic function. Media samples were collected daily and were stored at 4° C. for subsequent analysis for albumin content. Albumin content was measured by an enzyme-linked immunsorbent assay (ELISA) as described in Dunn et al., Biotechnol, Prog., vol.7, pp.237-245 (1991).

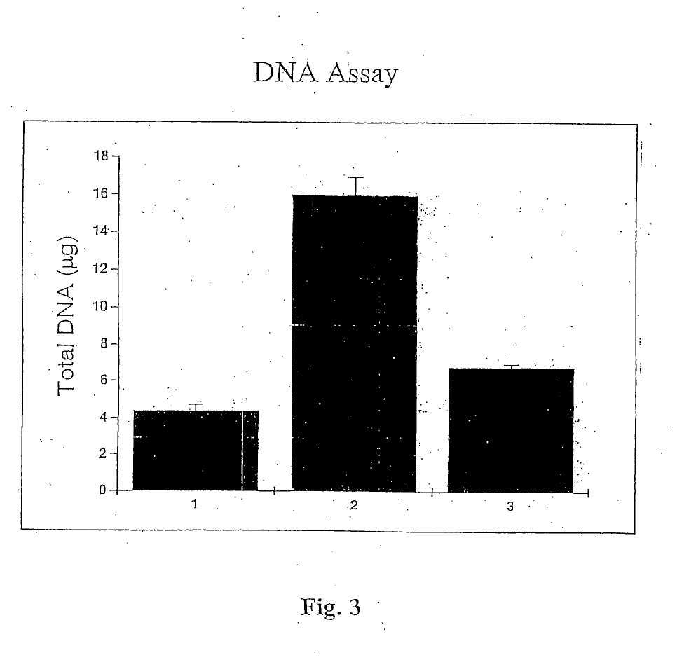

FIG. 1 shows albumin production by hepatocytes grown on a double gel substrate, liver basement membrane, or adsorbed collagen. DNA measurements indicated that 3 times more cells were present on the double gel substrate than on LBM (see FIG. 3). Accordingly, on a per cell basis, hepatocytes grown on LBM produce about the same amount of albumin as hepatocytes grown on the double gel substrate (positive control). Thus, hepatocytes grown on LBM exhibit liver synthetic function. In addition, albumin synthesis is maintained or increases when hepatocytes are grown on LBM. In contrast, albumin synthesis declines when hepatocytes are grown in conventional culture (i.e., on adsorbed collagen).

### EXAMPLE 8

Urea Assay

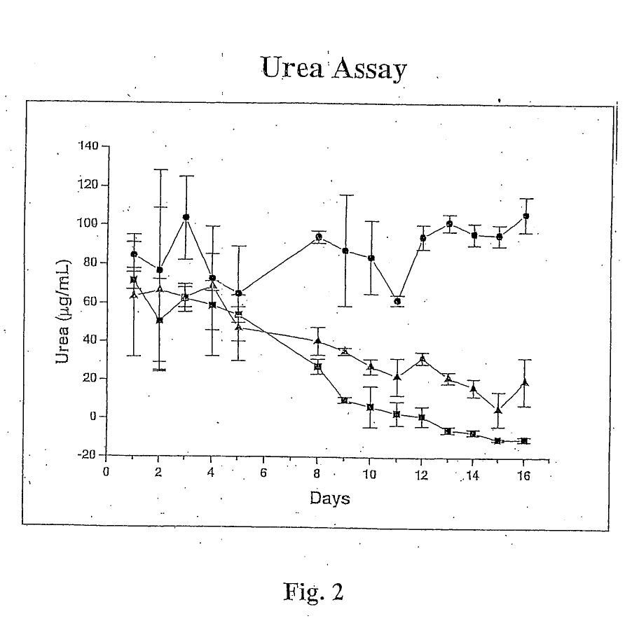

Hepatocytes were isolated and cultured as described in Example 7. A urea assay was performed on hepatocytes grown on a double gel substrate (circles in FIG. 2), liver basement membrane (triangles in FIG. 2), or on adsorbed collagen (squares in FIG. 2). The urea assay is a marker of liver metabolic function. Urea content in media samples collected daily as described in Example 6 was measured using a commercially available kit (Sigma Chemical Co., Kit No. 535-A).

FIG. 2 shows urea production by hepatocytes grown on the various substrates. On a per cell basis (see FIG. 3), hepatocytes grown on LBM produce about the same amount of urea as hepatocytes grown on the double gel substrate (positive control). Thus, hepatocytes grown on LBM exhibit liver metabolic function.

### EXAMPLE 9

DNA Assay

Hepatocytes were isolated and cultured as described in Example 7. A DNA assay was performed on hepatocytes grown on a double gel substrate (bar 2 in FIG. 3), liver basement membrane (bar 3 in FIG. 3), or adsorbed collagen (bar 1 in FIG. 3). The DNA assay was performed as described in Biotechnol Prog., vol. 14, pp. 378-387 (1998). As shown in FIG. 3, about 3 times more hepatocytes were present on the double gel substrate than on LBM. Viability of hepatocytes was also determined using dimethylthiazol-diphenyltetrazolium bromide cleavage to an insoluble purple product (MTT, Sigma-Aldrich, St. Louis, Mo.), extraction in 50% isoproponal/50% DMSO and measurement of absorbance at 570 mn.

### EXAMPLE 10

Cytochrome P450 Activity Assay

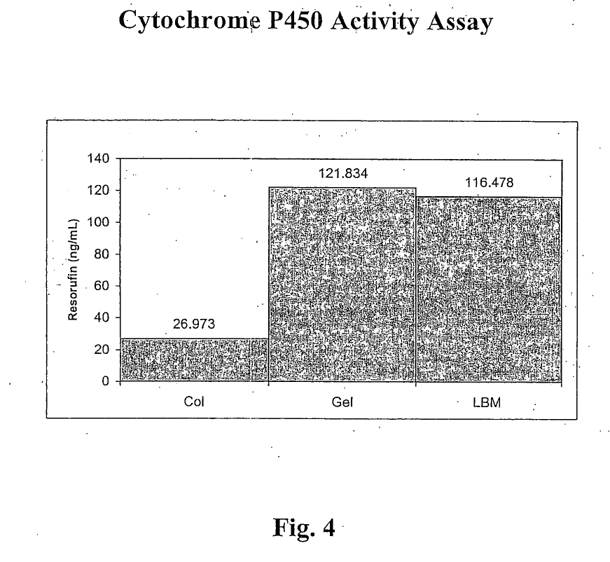

Hepatocytes were isolated and cultured as described in Example 7. A cytochrome P450 activity assay was performed on hepatocytes grown on a double gel substrate (bar labeled “gel” in FIG. 4), liver basement membrane (bar labeled “LBM” in FIG. 4), or on adsorbed collagen (bar labeled “col” in FIG. 4). The cytochrome P450 activity assay is a marker of liver metabolic function. Cytochrome P450 IA1 activity was determined by measuring cytochrome P450-dependent resorufin o-dealkylase activity essentially as described in detail in Behnia, et al., Tissue Engineering, vol. 6, pp. 467-479 (2000).

FIG. 4 shows cytochrome P450 activity, at day 48 after initiation of cell culture, for hepatocytes grown on the various substrates. On a per cell basis hepatocytes grown on LBM have at least, if not greater than, the level of cytochrome P450 activity that is observed for hepatocytes grown on the double gel substrate (positive control). Thus, hepatocytes grown on LBM exhibit liver metabolic function comparable to hepatocytes grown on the double gel substrate (the positive control) based on cytochrome P450 activity.

### EXAMPLE 11

Repair of Liver Tissue with LBM

Surgical methods for replacing damaged or diseased liver tissue with graft materials are known to the skilled artisan. Proper surgical procedures will be followed to anesthetize and prepare the patient for sterile surgery. The damaged or diseased site will be repaired with a multilaminate or a single-layer LBM graft. The LBM graft will be attached to the normal liver tissues using art-recognized techniques such as suturing and attachment of the graft with staples. The LBM graft composition will induce repair of damaged or diseased liver tissue in vivo.

### EXAMPLE 12

Hepatocvte Isolation

For experiments with similar results, rat hepatocytes were isolated from 2-3 month old adult female Lewis rats (Charles River Laboratories) weighing 180-200 g by collagenase perfusion and purified by filtration and Percoll centriftigation as described in Dunn, J. C., et al., Faseb J, 1989, 3(2): pp. 174-7. Normally, 200 to 300 million cells were isolated with an 85 to 95 % viability determined by a trypan blue exclusion dye. Culture medium was Dulbecco's modified eagle medium (DMEM, Invitrogen) supplemented with 10% fetal bovine serum (Sigma), insulin, glucagon, and hydrocortisone (UCSD Pharmacy).

### EXAMPLE 13

Preparation of LBM

For experiments with similar results, 5 mm thick sheets were prepared from whole porcine liver. The tissue was immersed in distilled water for 24 hours at 4° C. to lyse resident cells. After 24 hours, the distilled water was replaced by 0.05% ammonium hydroxide solution containing 0.5% Triton X-100 for 72 hours. The decellularized tissue was subsequently equilibrated with phosphate buffered saline at 4° C. The material was then lyophilized for 24 hours and subsequently sterilized with 2.0 Mrad gamma irradiation.

### EXAMPLE 14

Hepatocyte culture

For experiments with similar results, hepatocytes were cultured under three different conditions. Hepatocytes were cultured on LBM membranes, between two layers of collagen I gel (double gel; DG), or on adsorbed collagen I (AC) on tissue-culture polystyrene. LBM membranes were rehydrated in DMEM for 20 minutes prior to cell seeding and the membranes were held stationary in P-60 dishes by stainless steel inserts. The membrane covered ˜95% of the petri dish surface. For double gel cultures, concentrated DMEM (10×) was rapidly mixed with 1 mg/mL of rat-tail collagen I, prepared as described in Dunn, J. C., et al., Faseb J, 1989, 3(2): pp. 174-7, at a concentration of 9:1 (v/v) and kept on ice. The solution formed a gel upon incubation at 37° C. for 45 minutes. Adsorbed collagen surfaces were prepared by incubation of the polystyrene surface with 110 μg /mL of collagen I in ddH₂O for 45 minutes. Cultures were seeded with 1.5×10⁶ primary hepatocytes in 3 mL of media. The following day, unattached cells were removed by washing with 3 mL of media. Double gel cultures were overlaid with a second layer of gel followed by the addition of 3 mL of media. Media was replaced daily and spent media was stored at 4° C. for further analysis.

### EXAMPLE 15

Microscopy

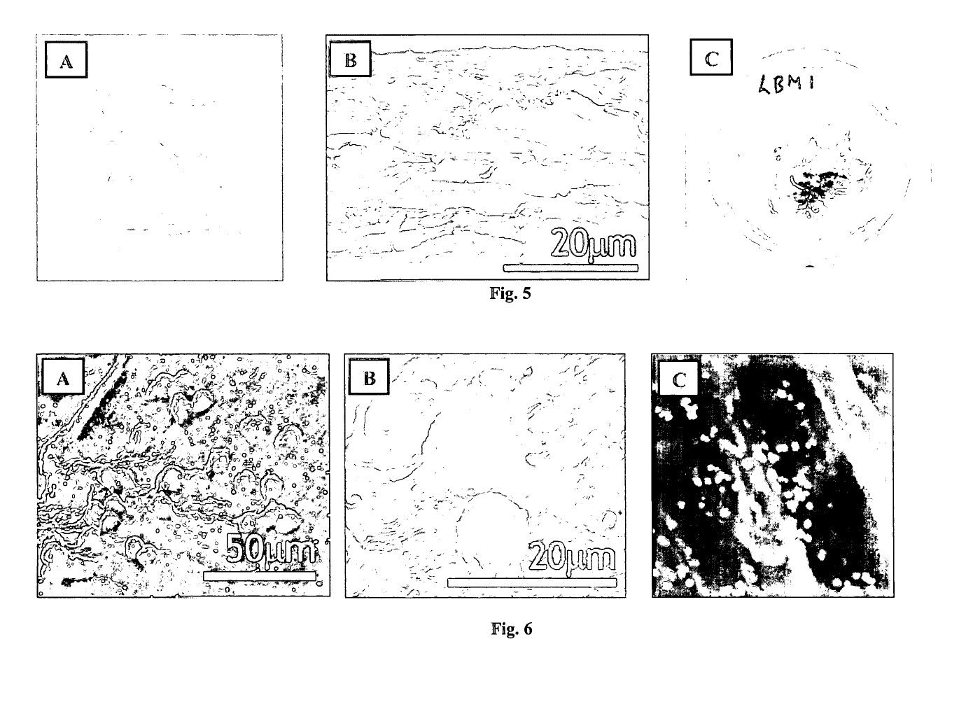

For the experiments shown in FIGS. 5 and 6, measurements of projected surface area were performed by phase contrast microscopy using a Nikon Diaphot microscope, captured with a SPOT camera, and analyzed with Metamorph Image Analysis software. Twenty cells were measured for each condition. For fluorescence imaging, cultures were washed with DMEM and incubated with 1 μg/mL of 5-(and-6)-(((4-chloromethyl)benzoyl)amino) tetramethylrhodamine (CMTMR) in DMEM for 30 minutes. Afterward, cultures were washed three times in 10 mM PBS, pH =7.4 and fixed with 4% paraformaldehyde in PBS for 20 minutes. Hepatocytes were observed at ex/em of 541/565 nm. For scanning electron microscopy, cultures were fixed using 4% paraformaldehye, dehydrated, and sputtered with a 100 nm layer of gold-palladium (50 mTorr, Anatech), and imaged with an SEM (Cambridge SEM 360) at an EHT of 20.0 KV.

1. A method for inducing the repair of damaged or diseased liver tissue in a patient in need thereof, said method comprising the step of administering to the patient a graft composition comprising basement membrane tissue of a warm-blooded vertebrate in an amount effective to induce the repair of the liver tissue at the site of administration of the graft composition.

2. The method of claim 1 wherein the graft composition is fluidized and is administered by injection into the patient.

3. The method of claim 1 wherein the basement membrane is in sheet form and the graft composition is administered by surgically implanting the graft composition into the patient.

4. The method of claim 1 wherein the basement membrane is in the form of a gel.

5. The method of claim 1 wherein the basement membrane is in powder form.

6. The method of claim 1 wherein the graft composition is a multilayered graft composition formed from two or more layers of liver basement membrane.

7. The method of claim 6 wherein the layers of liver basement membrane have a thickness of up to about 2000 μm.

8. The method of claim 6 wherein the graft composition is formed as a multilayered homolaminate graft composition.
